(19) United States
(12) Patent Application Publication
(10) Pub. No.: US 2026/0191568 A1
(43) Pub. Date: Jul. 9, 2026

(54) IMPLANTABLE STABILIZATION DEVICE

(71) Applicant: Acumed LLC, Hillsboro, OR (US)

(72) Inventors: Devin Collins, Loop Lakeland, FL (US); William Mack, Portland, OR (US); Kenny Chinn, Hillsboro, OR (US); Steven P. Horst, Dayton, OR (US)

(21) Appl. No.: 19/424,870

(22) Filed: Dec. 18, 2025

Related U.S. Application Data
(60) Provisional application No. 63/741,682, filed on Jan. 3, 2025.

(51) Int. Cl. A61B 17/72; A61B 17/17

(52) U.S. Cl. CPC A61B 17/72; A61B 17/1717

(57) ABSTRACT

Provided herein are methods and systems for stabilizing a joint, such as the elbow. The system may include an intramedullary rod configured to be placed in a medullary canal of a bone, a pin, a linking member defining an aperture for receiving the pin, and a connector defining a first aperture and a second aperture, the first aperture configured to receive the linking member. The system may further include a screw extending through the second aperture of the connector and the aperture of the intramedullary rod. The system may be configured to be implanted into a patient.

### INCORPORATION BY REFERENCE TO ANY PRIORITY APPLICATIONS

This application claims benefit of U.S. Provisional Patent Application No. 63/741,682, filed Jan. 3, 2025 and entitled “IMPLANTABLE STABILIZATION DEVICE,” the disclosure of which is hereby incorporated by reference and made part of this specification as if set forth fully herein in its entirety. Any and all applications for which a foreign or domestic priority claim is identified in the Application Data Sheet as filed with the present application are hereby incorporated by reference under 37 CFR 1.57.

### BACKGROUND

Injury to the elbow, such as fractures, dislocations, and soft tissue injuries may result in damage to the collateral ligaments of the elbow. A surgeon may attempt to repair the collateral ligaments, but the elbow may be unstable during healing. A surgeon may use a stabilization device to temporarily support the loads on the elbow until the ligaments are fully healed. However, some current methods require immobilization of the elbow, which can lead to elbow stiffness. Other current methods include systems located external to the patient's skin, which may be uncomfortable for the patient or prone to infection.

There remains a need for a device that allows mobility of the elbow while also preventing the drawbacks associated with current devices.

### SUMMARY

The present disclosure provides new and innovative devices, systems, and methods for stabilizing a joint that avoids problems associated with the current solutions, such as irritation, increased infections, and joint immobility. The devices, systems, and methods provided herein use an intramedullary rod that is configured to be inserted into a bone. The intramedullary rod may be configured to connect to a pin located through the axis of rotation of the joint. The system may improve comfort while still allowing patient mobility during recovery. Additionally, the system may be adjustable so that it can be used with different patients and on different sides of the joint.

Another aspect of the present disclosure provides an axis guide and methods for determining the rotation axis of the elbow. The axis guide may include a first arm, a second arm, and a pivot point connecting the first arm and the second arm. Each arm may include a radiopaque portion extending through a central axis. A user may pivot the first arm about the second arm until the central axis of each arm aligns with certain anatomical landmarks of the elbow. The rotation axis of the elbow may be at the intersection of the central axis of the first arm and the central axis of the second arm when each arm is in the desired location.

Additional features and advantages of the disclosed methods are described in, and will be apparent from, the following Detailed Description and the Figures.

### BRIEF DESCRIPTION OF THE DRAWINGS

FIG. 1 illustrates a system for joint stabilization according to an example of the present disclosure.

FIG. 2 illustrates a system for joint stabilization according to FIG. 1 with a modified hinge.

FIG. 3 illustrates a system for joint stabilization according to FIG. 1 with a flexible linking member.

FIG. 4 illustrates a flexible linking member according to an example of the present disclosure.

FIG. 5 illustrates a guide for determining a rotation axis according to an example of the present disclosure.

FIG. 6 illustrates a guide for determining a rotation axis according to an example of the present disclosure in use.

### DETAILED DESCRIPTION

The present disclosure is directed to devices, systems and methods for stabilizing a joint. Stabilizing the joint may include preventing movement of the joint that would otherwise be prevented by natural ligaments within the joint. In some embodiments, the devices, systems and methods may stabilize the elbow joint. For example, the system disclosed herein may prevent varus or valgus instability of the elbow joint. In some embodiments, the devices and systems disclosed herein may be configured to be fully implantable or internal. Fully implantable or internal may mean the components of the device and system are located under the skin after implantation. The devices and systems disclosed herein may allow mobility of the elbow while in use.

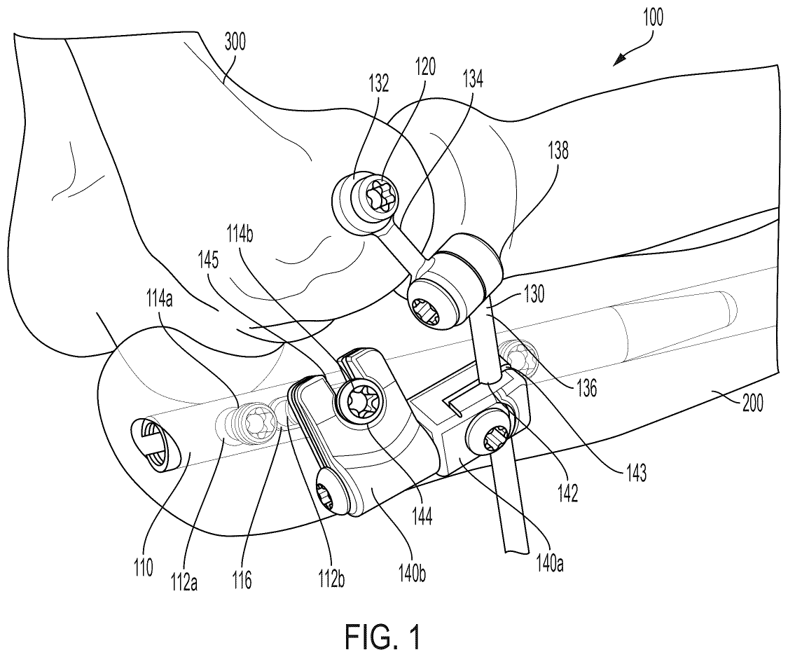

FIGS. 1-3 illustrate a system 100 for stabilizing a joint according to an example of the present disclosure. In a preferred embodiment, the system 100 includes an intramedullary rod 110. As illustrated in the depicted embodiments, the intramedullary rod 110 may be configured to be inserted into the medullary canal of a bone, such as the ulna 200. The intramedullary rod 110 may provide internal fixation of the system 100 with the joint. The use of the intramedullary rod 110 may reduce irritation to the patient.

In some embodiments, intramedullary rod 110 includes an elongate body. The elongate body may be cylindrical, conical, rectangular, square, or any desired shape. The width of the body may taper along a portion of the length. Additionally or alternatively, an end of the intramedullary rod 110 may include a rounded or pointed tip. The tapering and/or the tip may aid in insertion of the rod 110 within the intramedullary canal. While the depicted embodiments illustrate a smooth exterior surface of the intramedullary rod 110, in some embodiments, the rod 110 may include one or more threads along a portion or along the whole length of the rod 110.

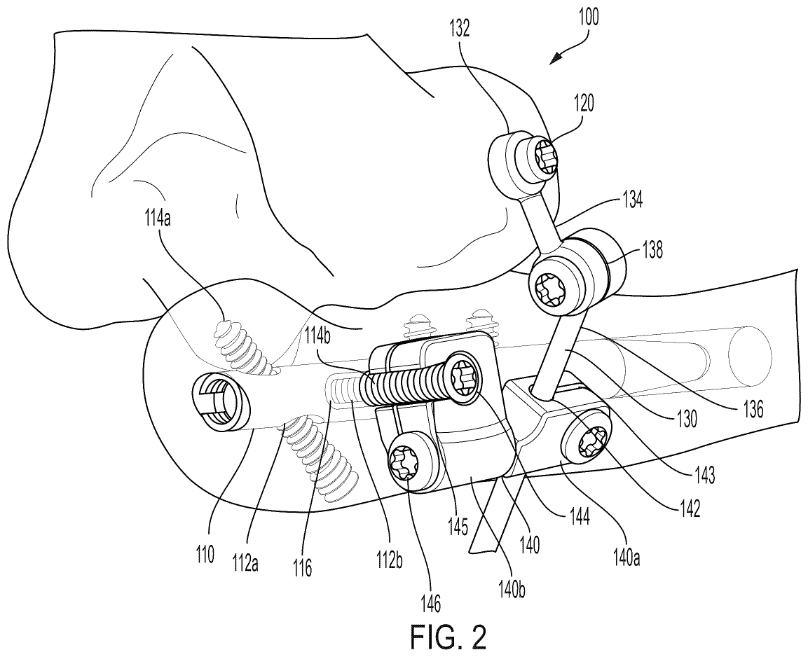

In some embodiments, the intramedullary rod 110 may include one or more apertures 112a, 112b. The one or more apertures 112a may be configured to receive a fixation device 114a, such as a screw, to secure the rod 110 within the bone. In some embodiments, the one or more apertures 112b may be configured to receive a fixation device 114b, such as a compression screw, to secure the rod 110 to the remainder of the system 100, which will be described in more detail herein. The one or more apertures 112a, 112b may be perpendicular to a central axis of the intramedullary rod 110. Additionally or alternatively, as shown in FIG. 2, the one or more apertures (i.e. aperture 112a) may be traverse to a central axis of the intramedullary rod 110.

In some embodiments, the one or more apertures of the intramedullary rod may include a slot 112b. The slot 112b may include a lining 116 with a material configured to engage the fixation device 114b (e.g. one or more threads of a screw). For example, the lining 116 of the slot 112b may secure the fixation device 114b in a set position along the length of the slot 112b. In some embodiments, the lining 116 of the slot 112b includes a PEEK insert. This may allow the system 100 to be adjustable based on the desired stabilization position of the joint. An adjustable system 100 may be advantageous because it may be used with many different patients and does not require customization.

The stabilization system 100 may further include a pin 120. The pin 120 may include a head and a shaft. In some embodiments, the head has a greater width than the shaft. The head may include a recess for engaging with a driver, such as a hexalobe or any other desired driver type. In some embodiments, the shaft of the pin 120 may include a smooth exterior surface (i.e. non-threaded). In some embodiments, at least a portion of the pin 120 (e.g. the head and/or the shaft) may include a thread. In some embodiments, the pin may include a diameter of about 2.0 mm, about 2.5 mm, or about 3 mm.

The pin 120 may be configured to be inserted into an axis of rotation of the joint. For example, as shown in the depicted embodiments, the pin 120 may be inserted into the axis of rotation of the elbow, such as the distal end portion of the humerus 300. The joint stabilization system 100 may allow the elbow to rotate about the pin 120 to provide flexion of the elbow during healing.

In a preferred embodiment, the stabilization system 100 includes components connecting the intramedullary rod 110 to the pin 120. In some embodiments, the system 100 includes a linking member 130. The linking member 130 may include an aperture 132 for receiving the pin 120. In some embodiments, the head of the pin 120 is configured to fit within the aperture 132 of the linking member 130. The aperture 132 of the linking member 130 may include threading on an internal surface configured to engage a thread (either on the head or the shaft) of the pin 120.

In some embodiments, the linking member 130 may include a first portion 134, a second portion 136, and a pivot point 138 between the first and the second portion 134, 136. The first portion 134 and the second portion 136 may each include an aperture for receiving a pin or a screw to act as the pivot point 138. The first and second portion may be able to move about the pivot point until the screw is advanced to secure the two portions 134, 136 in place. In some embodiments, the aperture of the first portion 134 may include a set of teeth configured to engage a set of teeth of the aperture of the second portion 136. The teeth of the apertures can secure the first and second portion 134, 136 in the correct orientation for stabilization (e.g. once the screw is advanced through the apertures).

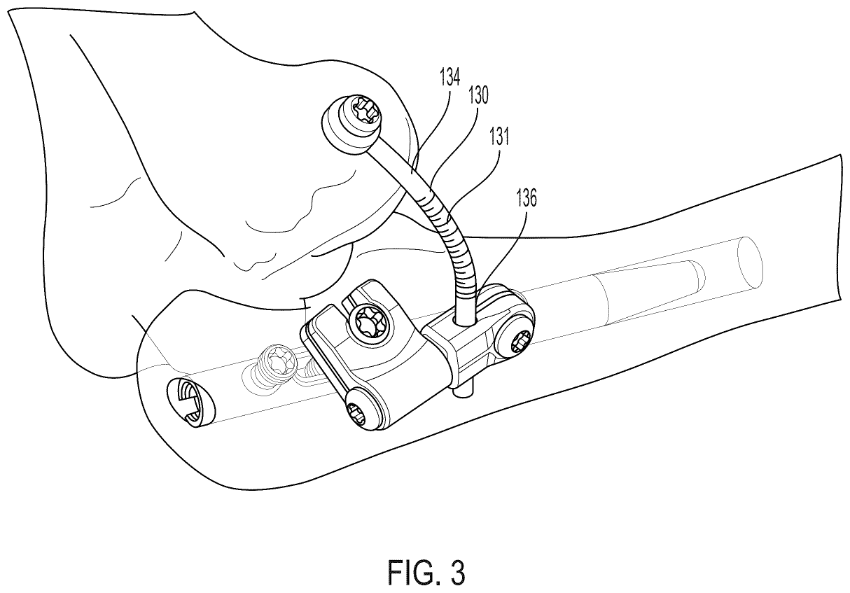

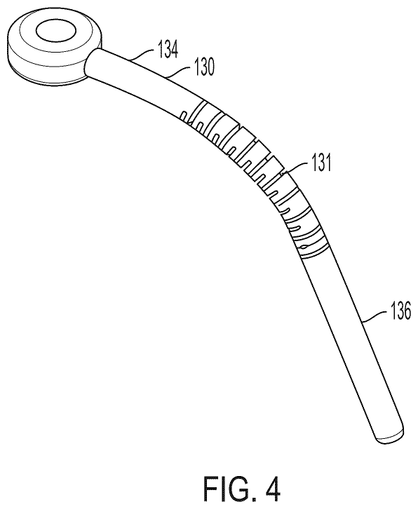

In some embodiments, the linking member 130 may be made of a rigid biocompatible material (e.g. metal alloys (titanium alloys, cobalt chromium alloys, stainless steel, etc.)). As depicted in FIGS. 3 and 4, the linking member 130 may include a flexible portion 131 between the first portion 134 and the second portion 136. For example, the flexible portion 131 may include one or more cuts or cutouts that allow an otherwise rigid material to bend or “flex”. In some examples, the one or more cuts may traverse the entire diameter or a partial diameter of the linking member 130. In some examples, the flexible portion 131 may include a cannulation through the central axis of the linking member 130. The one or more cuts or cutouts may extend from the outside surface of the linking member 130 through the inside surface of the cannulation.

The one or more cutouts may include one or more helical cuts, such as one (two, three, four . . . ) continuous helical cut(s), around the flexible portion 131 of the linking member 130. It can be appreciated that other laser cutting techniques may be used to impart a flexible quality to a portion of the linking member 130. For example, the helical cut may include a straight helical cut, or the helical cut may include a series of patterns (such as dove tail cuts, sinusoidal patterns, etc.) along the helical path. In some examples, the one or more cuts may include one or a plurality of dovetail cuts, sinusoidal patterns or straight cuts surrounding a partial or a full circumference of the linking member 130.

In some embodiments, the flexible portion 131 may include a portion of the linking member 130 with a smaller diameter or width than the remainder of the linking member 130. The portion of the linking member with the smaller diameter or width may be solid but could still impart flexibility to the linking member 130. In some embodiments, the smaller diameter or width may be between 10%-50%, such as 10%, 20%, 30%, 40%, or 50% of the diameter or width of the remainder of the linking member 130.

The system may include a connector 140 for connecting the linking member 130 and/or the pin 120 to the intramedullary rod 110. In some embodiments, the connector 140 may include at least a first aperture 142 and a second aperture 144. The first aperture 142 may be configured to receive the linking member 130. In some embodiments, the first aperture 142 includes a slot 143 extending through a side of the connector 140. As shown in FIG. 1, the slot 143 may be configured to receive the linking member 130 so that the linking member 130 is side loaded into the first aperture 142. In some embodiments, a portion of the linking member 130, such as the second portion 136, is configured to be inserted into the first aperture 142 through a top or bottom surface of the first aperture 142 as shown in FIG. 2.

In some embodiments, the connector 140 includes an additional aperture traversing through the slot 143. The aperture may be configured to receive a screw. Tightening of the screw may compress the slot 143 and the first aperture 142 around the linking member 130. The linking member 130 may be fixed within the first aperture 142 so that the system 100 is fixed in a desired orientation for stabilization.

The second aperture 144 may be configured to receive a fixation device 114b, such as a compression screw. The fixation device 114b may be configured to be inserted through the second aperture 144 and into an aperture 112b of the intramedullary rod 110, such as slot 112b. As described previously, in some embodiments, the slot 112b of the intramedullary rod 110 may include a lining 116 configured to secure the fixation device 114b along the length of the slot 112b (e.g. by engaging a thread of a fixation device 114b).

As shown in FIG. I, the second aperture 144 may include a slot 145 extending from the second aperture 144 through a top edge of the connector 140. As shown in FIG. 2, the slot 145 may extend from the second aperture 144 through a side edge of the connector 140. This may allow the fixation device 114b to be top or side loaded onto the connector 140. For example, the fixation device 114b may be loaded through the slot 145 and into the second aperture 144 after the fixation device 114b has been screwed into the bone.

As shown in the depicted embodiments, the connector 140 may include a first portion 140a and a second portion 140b. The first portion 140a may be able to articulate about the second portion 140b. In some embodiments, the second portion 140b includes a pin or screw 146 extending therethrough and into the first portion 140a or the first portion 140a includes a pin or screw 146 extending therethrough and into the second portion 140b. The first and second portions 140a, 140b may articulate about the screw or pin 146. In some embodiments, the first aperture 142 is in the first portion 140a and the second aperture 144 is in the second portion 140b.

Another aspect of the present disclosure is a method of stabilizing a joint using the joint stabilization system disclosed herein. The method may may include installing the intramedullary rod 110 into an intramedullary canal of a bone, such as the ulna 200. In some embodiments, the rod 110 is installed using a targeting guide. After insertion into the medullary canal, the intramedullary rod 110 may be secured to the bone by inserting one or more fixation devices 114a into the bone and through an aperture 112a of the intramedullary rod 110. ln some embodiments, the one or more fixation devices 114a may be inserted into the rod 110 from the lateral side of the ulna to the medial side of the ulna as shown in FIG. 1 or vice versa.

The method may further include determining an axis of rotation of the elbow joint, which will be described in more detail herein. The pin 120 may be drilled into the axis of rotation of the elbow, such as on a distal end of the humerus 300. The pin 120 may be coupled to a portion of the linking member 130 so that the linking member 130 can pivot about the pin 120. This allows flexion of the elbow while still maintaining stabilization typically provided by the collateral ligaments.

In some embodiments, the pin 120 is coupled to the linking member 130 prior to inserting the pin 120 into the axis of rotation. For example, the pin 120 may couple to the linking member 130 by inserting the pin 120 into an aperture of the linking member 130. In some embodiments, the linking member 130 may be coupled to the pin 120 after the pin 120 is inserted into the rotation axis. For example, the linking member 130 may clip onto or otherwise secure onto the linking member 130 after the pin 120 is installed.

The method may include securing the connector 140 to the intramedullary rod 110. Securing the connector 140 to the intramedullary rod 110 may occur before or after inserting the pin 120 into the bone. In some embodiments, the connector 140 is secured to the rod 110 by inserting a fixation device 114b, such as a compression screw, through an aperture 144 of the connector 140 and an aperture (e.g. slot 112b) of the rod 110. The connector 140 may be secured to the rod 110 by loading the fixation device 114b through a slot 145 in the connector 140 after the fixation device 114b has been drilled through the bone into an aperture 112b of the intramedullary rod 110. The connector 140 may be secured to the lateral or the medial side of the ulna 200. This may reduce irritation to the patient compared to systems with a protruding surface on the posterior surface the ulna 200.

The method may include adjusting the joint into a desired position for stabilization. The linking member 130 may be adjusted so that securement of the linking member 130 to the connector 140 holds the elbow in the desired position. For example, the method may include adjusting the first and second portions 134, 136 about the pivot point 138 until a desired angle is reached for stabilization. Once the desired angle is reached, the first and second portions 134, 136 may be secured in place by tightening a screw through apertures at the pivot point 138. In some embodiments, if the linking member 130 has a flexible portion 131 as shown in FIGS. 3 and 4, the method may include flexing the flexible portion 131 of the linking member 130 until a desired orientation is reached.

A portion of the connector 140 may be adjusted to receive the linking member 130 at the desired angle. For example, as shown in the depicted embodiment, the first portion 140a of the connector 140 may be articulated relative to the second portion 140b so that the aperture 142 is at a desired angle to receive the linking member 130. In an alternative embodiment, the second portion 136 of the linking member 130 may be within the first aperture 142 of the connector 140 and the first portion 134 of the linking member 130 may be coupled to the pin 120. A user may articulate the first portion 140a of connector 140 so that the apertures of the first portion 134 and the second portion 136 of the linking member 130 align. A user may then secure the first portion 134 and the second portion 136 together.

The linking member 130 may be secured to the connector 140 by inserting an end of the linking member 130, such as the second portion 136, into the first aperture 142 of the connector 140. In some embodiments, the linking member 130 is secured to the connector 140 by sliding the linking member 130 through a slot 145 in a side portion of the connector 140 and into the aperture 142 of the connector 140. A user may translate the linking member 130 through the aperture 142 until the linking member 130 is a desired length for stabilization. The screw may then be tightened to compress the aperture 142 in the connector 140 around the linking member 130 to secure the linking member 130 within the aperture 142 of the connector 140. The linking member 130 may be trimmed after a desired positioning has been reached. This allows adjustability of the system while still avoiding irritation from the portion of the linking member 130 extending from the connector 140.

While the depicted embodiments show the stabilization system 100 on the lateral side of the elbow joint, in some embodiments, the stabilization system 100 may be implanted on the medial side of the elbow joint. In some embodiments, the stabilization system is configured to be used on both the lateral and medial side of the ulna 200. This prevents the need for two different stabilization systems, one for the lateral side and one for the medial side. In some embodiments, the stabilization system 100 is configured to stabilize both the lateral and medial sides at the same time. For example, the medial side of the ulna may include a connector, a linking member, and a pin to be used with intramedullary rod 110 in addition to the connector 140, the linking member 130, and the pin 120 shown on the lateral side. In some embodiments, pin 120 may include a cannulation or recess in the shaft configured to receive a pin from the medial side of the humerus 300. In some embodiments, the cannulation may include threads configured to mate with threads of the pin from the medial side of the system.

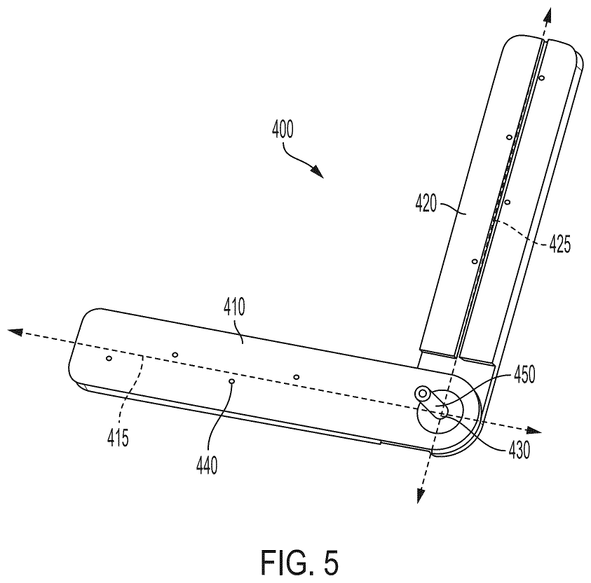

FIG. 5 illustrates an axis guide 400 according to an example of the present disclosure. In some embodiments, the axis guide 400 may be used to determine the rotation axis of the elbow. The axis guide 400 may include a first arm 410, a second arm 420, and a pivot point 430 connecting the first arm 410 to the second arm 420. The central axis of each arm may intersect at the pivot point 430. The axis 415 of the first arm 410 and the axis 425 of the second arm 420 may extend through the center of and/or parallel to a side edge of each respective arm 410, 420. In some embodiments, the axis guide 400 is made of a radiolucent material.

In some embodiments, the axis guide 400 may include one or more radiopaque portions. The radiopaque portions may be used to align the axis guide 400 on the joint to determine the rotation axis. For example, the axis guide 400 may include a first radiopaque wire extending along an axis 415 of the first arm 410 and a second radiopaque wire extending along an axis 425 of the second arm 420. The wires may be formed integrally with the axis guide 400 or the wires may be separate components from the axis guide. For example, in some embodiments, a wire is configured to fit into a groove positioned along an axis 415,425 of the axis guide 400.

In some embodiments, the axis guide 400 includes one or more apertures 440. The one or more apertures 440 may be configured to receive a guide wire. The guide wire may hold the axis guide 400 in place during use. Additionally or alternatively, the axis guide 400 may include an aperture 450 for drilling a hole in the bone at the axis of rotation of the elbow. The aperture 450 for drilling the hole at the axis of rotation may be placed at the pivot point of the axis guide (i.e. the intersection of the first central axis 415 and the second central axis 425). The drilled hole may be the insertion site of the pin 120 of the stabilization system 100. The axis guide 400 may also include a built-in drill guide for drilling the hole. The built-in drill guide may also measure the hole to determine the proper length of the pin.

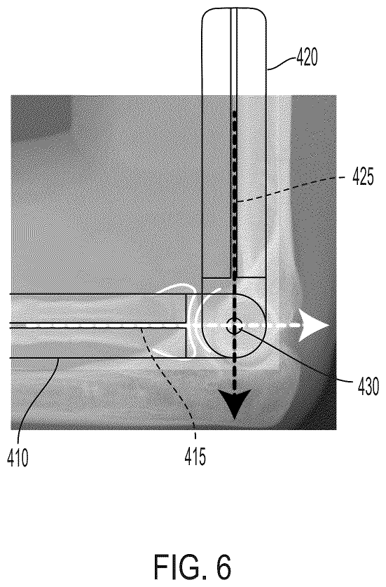

FIG. 6 illustrates an example method for determining the axis of rotation of the elbow using the axis guide 400 disclosed herein. As shown in FIG. 6, the user may position the elbow joint into a “perfect lateral” position as understood by one skilled in the art. For example, the perfect lateral position may include positioning the elbow joint at a substantially 90° flexion angle. Additionally, the perfect lateral position may include positioning the hand and wrist in a true lateral position, such as with the thumb up.

A user may position and adjust the axis guide 400 on the elbow joint. The method may include aligning an axis 415 of the first arm 410 along the radiocapitellar line. The method may further include aligning an axis 425 of the second arm 420 along the anterior humeral line. Aligning the axis may include aligning the radiopaque portion of the first arm 410 through the center of the radial head and aligning the radiopaque portion of the second arm 420 on the anterior humeral line as shown in FIG. 6. For example, a user may pivot the first arm 410 about the second arm 420 until the axis guide 400 is in the desired position. This can be done using radiographic imaging. The axis of rotation of the elbow may be the intersection of the central axis 415 of the first arm 410 and the central axis 425 of the second arm 420.

In some embodiments, once the axis guide 400 is in the desired position, the method may include fixing the axis guide 400 with one or more guidewires. A hole may be drilled through an aperture 450 of the axis guide 400 at the rotation axis.

### EMBODIMENTS

Various aspects of the subject matter described herein are set out in the following numbered embodiments:

Embodiment 1. An internal joint stabilization system comprising: an intramedullary rod configured to be placed in a medullary canal of a bone, the intramedullary rod defining an aperture; a pin; a linking member defining an aperture for receiving the pin; a connector defining a first aperture and a second aperture, the first aperture configured to receive the linking member; and a screw extending through the second aperture of the connector and the aperture of the intramedullary rod.

Embodiment 2. The system of embodiment 1, wherein the first aperture of the connector comprises a slot extending through an end portion of the connector.

Embodiment 3. The system of embodiment 2, wherein the connector defines a third aperture and wherein the third aperture of the connector is configured to receive an additional screw, wherein the additional screw is configured to compress the slot to secure the linking member with the connector.

Embodiment 4. The system of any one of embodiments 1-3, wherein the connector comprises a first connector portion and a second connector portion, wherein the first connector portion is configured to articulate about the second connector portion.

Embodiment 5. The system of embodiment 4, wherein the first aperture of the connector is on the first connector portion and wherein the second aperture of the connector is on the second connector portion.

Embodiment 6. The system of any one of embodiments 1-5, wherein the linking member comprises a first link portion, a second link portion, and a pivot point between the first link portion and the second link portion.

Embodiment 7. The system of any one of embodiments 1-6, wherein the linking member is comprised of a rigid material and wherein the linking member comprises one or more groves configured to allow the linking member to flex.

Embodiment 8. The system of any one of embodiments 1-7, wherein the aperture of the intramedullary rod comprises a slot.

Embodiment 9. The system of any one of embodiments 1-8, wherein the pin comprises a head and a shaft and wherein the head is configured to fit into the aperture of the linking member.

Embodiment 10. A method of stabilizing an elbow using a joint stabilization system, the joint stabilization system comprising: an intramedullary rod defining an aperture; and a connector defining a first aperture and a second aperture, wherein the method comprises: installing the intramedullary rod into an ulna of a patient, positioning the connector on the ulna, securing the connector to the intramedullary rod by inserting a screw through the second aperture of the connector and the aperture of the intramedullary rod, inserting a pin into a humerus and into an axis of rotation of the elbow, wherein the pin is coupled to a linking member, and securing the linking member in the first aperture of the connector.

Embodiment 11. The method of embodiment 10, wherein the connector comprises a first connector portion and a second connector portion, the first connector portion configured to articulate about the second connector portion, and wherein the method further comprises articulating the first connector portion about the second connector portion to achieve a desired angle of the linking member.

Embodiment 12. The method of any one of embodiments 10-11, wherein the first aperture comprises a slot in an end portion of the connector, and wherein the securing the linking member in the first aperture of the connector comprises sliding the linking member into the slot.

Embodiment 13. The method of embodiment 12, wherein the connector further defines a third aperture, and wherein the securing the linking member in the first aperture of the connector further comprises tightening a screw in the third aperture of the connector to compress the slot.

Embodiment 14. The method of any one of embodiments 10-13 further comprising trimming the linking member after securing the linking member in the first aperture of the connector.

Embodiment 15. The method of any one of embodiments 10-14 further comprising determining the axis of rotation of the elbow before inserting the pin into the humerus.

Embodiment 16. The method of embodiment 15, wherein the determining the axis of rotation of the elbow comprises: positioning an axis guide on an elbow joint of the patient, wherein the axis guide comprises: a first arm, a second arm, and a pivot point connecting the first arm and the second arm, wherein the first arm and the second arm comprise a radiopaque wire extending along an axis, wherein the axis is defined by a line that intersects the pivot point and is parallel to an edge of the first arm and the second arm, respectively; pivoting the first arm about the pivot point until the radiopaque wire of the first arm aligns with a radiocapitellar line of a radius; and pivoting the second arm about the pivot point until the radiopaque wire of the second arm aligns with an anterior humeral line of the humerus, wherein the axis of rotation is at the pivot point.

Embodiment 17. The method of embodiment 16, wherein the axis guide defines an opening at the pivot point, and wherein the method further comprises drilling a hole through the opening.

Embodiment 18. An axis guide configured to find a center of rotation, the axis guide comprising: a first arm, a second arm, and a pivot point connecting the first arm and the second arm, wherein the axis guide is made out of a radiolucent material, and wherein the first arm and the second arm comprise a radiopaque wire extending along an axis, wherein the axis defined by a line that intersects the pivot point and is parallel to an edge of the first arm and the second arm, respectively.

Embodiment 19. The axis guide of embodiment 18 defining one or more apertures configured to receive one or more guide wires.

Embodiment 20. The axis guide of any one of embodiments 18-19, wherein the pivot point defines an opening configured to receive a drill.

Embodiment 21. A method of determining an axis of rotation of an elbow, the method comprising: positioning an axis guide on an elbow joint of a patient, wherein the axis guide comprises: a first arm, a second arm, and a pivot point connecting the first arm and the second arm, wherein the first arm and the second arm comprise a radiopaque wire extending along an axis, wherein the axis intersects the pivot point and is parallel to an edge of the first arm and the second arm, respectively; pivoting the first arm about the pivot point until the radiopaque wire of the first arm aligns with a radiocapitellar line of a radius; and pivoting the second arm about the pivot point until the radiopaque wire of the second arm aligns with an anterior humeral line of a humerus, wherein the axis of rotation is at the pivot point.

Embodiment 22. The method of embodiment 21 further comprising capturing an x-ray of the elbow joint and the axis guide to determine if the radiopaque wire of the first arm aligns with the radiocapitellar line of the radius and the radiopaque wire of the second arm aligns with the anterior humeral line of the humerus.

Embodiment 23. The method of any one of embodiments 21-22, wherein the axis guide defines one or more apertures, and the method further comprises inserting one or more guidewires through the one or more apertures to secure the axis guide to the elbow joint.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

Additionally, in describing the components of the system of the present disclosure, there may be terms used like first, second, third, and fourth. These terms may be used for the purpose of differentiating one component from the other, but not to imply or suggest the substances, order, sequence, or number of the components.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. An internal joint stabilization system comprising:
an intramedullary rod configured to be placed in a medullary canal of a bone, the intramedullary rod defining an aperture;
a pin;
a linking member defining an aperture for receiving the pin;
a connector defining a first aperture and a second aperture, the first aperture configured to receive the linking member; and
a screw extending through the second aperture of the connector and the aperture of the intramedullary rod.

2. The internal joint stabilization system of claim 1, wherein the first aperture of the connector comprises a slot extending through an end portion of the connector.

3. The internal joint stabilization system of claim 2, wherein the connector defines a third aperture and wherein the third aperture of the connector is configured to receive an additional screw, wherein the additional screw is configured to compress the slot to secure the linking member with the connector.

4. The internal joint stabilization system of claim 1, wherein the connector comprises a first connector portion and a second connector portion, wherein the first connector portion is configured to articulate about the second connector portion.

5. The internal joint stabilization system of claim 4, wherein the first aperture of the connector is on the first connector portion and wherein the second aperture of the connector is on the second connector portion.

6. The internal joint stabilization system of claim 1, wherein the linking member comprises a first link portion, a second link portion, and a pivot point between the first link portion and the second link portion.

7. The internal joint stabilization system of claim 1, wherein the linking member is comprised of a rigid material and wherein the linking member comprises one or more grooves configured to allow the linking member to flex.

8. The internal joint stabilization system of claim 1, wherein the aperture of the intramedullary rod comprises a slot.

9. The internal joint stabilization system of claim 1, wherein the pin comprises a head and a shaft and wherein the head is configured to fit into the aperture of the linking member.

10. A method of stabilizing an elbow using a joint stabilization system, the joint stabilization system comprising:
an intramedullary rod defining an aperture; and
a connector defining a first aperture and a second aperture,
wherein the method comprises:
installing the intramedullary rod into an ulna of a patient,
positioning the connector on the ulna,
securing the connector to the intramedullary rod by inserting a screw through the second aperture of the connector and the aperture of the intramedullary rod,
inserting a pin into a humerus and into an axis of rotation of the elbow, wherein the pin is coupled to a linking member, and
securing the linking member in the first aperture of the connector.

11. The method of claim 10, wherein the connector comprises a first connector portion and a second connector portion, the first connector portion configured to articulate about the second connector portion, and wherein the method further comprises articulating the first connector portion about the second connector portion to achieve a desired angle of the linking member.

12. The method of claim 10, wherein the first aperture comprises a slot in an end portion of the connector, and wherein the securing the linking member in the first aperture of the connector comprises sliding the linking member into the slot.

13. The method of claim 12, wherein the connector further defines a third aperture, and wherein the securing the linking member in the first aperture of the connector further comprises tightening a screw in the third aperture of the connector to compress the slot.

14. The method of claim 10 further comprising trimming the linking member after securing the linking member in the first aperture of the connector.

15. The method of claim 10 further comprising determining the axis of rotation of the elbow before inserting the pin into the humerus.

16. The method of claim 15, wherein the determining the axis of rotation of the elbow comprises:
positioning an axis guide on an elbow joint of the patient, wherein the axis guide comprises:
a first arm,
a second arm, and
a pivot point connecting the first arm and the second arm,
wherein the first arm and the second arm comprise a radiopaque wire extending along an axis, wherein the axis is defined by a line that intersects the pivot point and is parallel to an edge of the first arm and the second arm, respectively;
pivoting the first arm about the pivot point until the radiopaque wire of the first arm aligns with a radiocapitellar line of a radius; and
pivoting the second arm about the pivot point until the radiopaque wire of the second arm aligns with an anterior humeral line of the humerus,
wherein the axis of rotation is at the pivot point.

17. The method of claim 16, wherein the axis guide defines an opening at the pivot point, and wherein the method further comprises drilling a hole through the opening.

18. An axis guide configured to find a center of rotation, the axis guide comprising:
a first arm,
a second arm, and
a pivot point connecting the first arm and the second arm,
wherein the axis guide is made out of a radio lucent material, and
wherein the first arm and the second arm comprise a radiopaque wire extending along an axis, wherein the axis defined by a line that intersects the pivot point and is parallel to an edge of the first arm and the second arm, respectively.

19. The axis guide of claim 18 defining one or more apertures configured to receive one or more guide wires.

20. The axis guide of claim 18, wherein the pivot point defines an opening configured to receive a drill.
